(19) United States
(12) Patent Application Publication
(10) Pub. No.: US 2025/0075238 A1
(43) Pub. Date: Mar. 6, 2025

(54) BIOSYNTHESIS OF HIERARCHICAL METAL ORGANIC FRAMEWORK-BACTERIAL CELLULOSE COMPOSITES

(71) Applicants: Northwestern University, Evanston, IL (US); The Hong Kong Polytechnic University, Kowloon (HK)

(72) Inventors: Omar K. Farha, Glenview, IL (US); John Xin, Kowloon (HK); Kaikai Ma, Evanston, IL (US); Yuk Ha Cheung, Kowloon (HK)

(21) Appl. No.: 18/726,607

(22) PCT Filed: Jan. 6, 2023
(86) PCT No.: PCT/US23/60199

§ 371 Date: Jul. 3, 2024

Related U.S. Application Data
(60) Provisional application No. 63/297,307, filed on Jan. 7, 2022.

(51) Int. Cl. C12P 19/04; C08K 5/56; C08L 1/02

(52) U.S. Cl. CPC C12P 19/04; C08K 5/56; C08L 1/02

(57) ABSTRACT

Composites of metal-organic framework particles and bacterial cellulose, methods of making the composites, and methods of using the composites in the hydrolysis of organic compounds are provided. The composites, which are aerogels comprising metal-organic framework particles embedded in a bacterial cellulose nanofiber network, are fabricated using a microbial synthesis strategy in which bacterial cellulose nanofiber is biosynthesized using a cellulose-producing bacteria in a fermentation medium in which metal-organic framework particles are dispersed.

### CROSS-REFERENCE TO RELATED APPLICATIONS

The present application claims priority to U.S. provisional patent application No. 63/297,307 that was filed Jan. 7, 2022, the entire contents of which are incorporated herein by reference.

### REFERENCE TO GOVERNMENT RIGHTS

This invention was made with government support under W911NF2020136 awarded by the Department of Defense, Department of the Army, Army Research Office and under HDTRA1-18-1-0003 awarded by the Department of Defense, Defense Threat Reduction Agency. The government has certain rights in the invention.

### BACKGROUND

Metal-organic frameworks (MOFs) are a class of crystalline porous framework materials constructed from the orderly coordination of metal ion nodes and organic ligands units. By varying the metal center and organic linkers, thousands of MOFs with enriched structure and chemical diversity have been developed and applied in gas storage and separation, heterogeneous catalysis, toxic chemical removal, and sensing. In their typical production, MOFs are obtained as fine powders with poor processability, which hinders the practicability and performance of MOFs in industrial applications. To this end, the incorporation of MOFs onto a supporting substrate has attracted great interest to tackle the shortcomings of MOF processing. Polymeric fibers with good processability can be integrated rationally with MOFs to yield materials with enhanced practicality and functionality. MOFs have been successfully deposited onto both natural fibers including cotton and silk as well as synthetic fibers such as polyurethane, polyethylene terephthalate, polyamide, polyacrylonitrile, polystyrene, polyvinylidene difluoride, and polypropylene. The prepared MOF/fiber composites possess integrated properties and performances which cannot be obtained from the individual components. MOF/fiber composites with good flexibility and permeability can be tailored into different shapes and forms for important liquid and gas-phase applications, such as catalytic detoxification of toxic warfare agents, toxic gas capture, healthcare, particulate matters removal, and water purification.

However, currently reported MOF/fiber composite syntheses have several challenges. Typically, the MOF/textile composites are produced at high temperatures, sometimes exceeding 100° C., using hazardous organic solvents such as N,N-dimethylformamide (DMF), N,N-dimethylacetamide (DMAC) and dimethyl sulfoxide (DMSO), which may be trapped in composites and pose a high risk to human health if they are used as protective gear like a mask filter. Moreover, the production of MOF/fiber composites using high temperatures and highly flammable organic solvents is challenging in industrial settings. Additionally, most polymeric fibers possess lower stability in polar organic solvents at elevated temperatures. For example, polyacrylonitrile, polystyrene, and polyvinylidene difluoride, which are used in the production of MOF/fiber composites, quickly dissolve in DMF at room temperature, thus disintegrating the entire MOF/fiber composite. Therefore, more environmentally friendly production methods are needed to produce a MOF-based composite with good stability.

### SUMMARY

Composites of MOFs and BC (MOF/BC composites), methods of making the composites, and methods of using the composites in the hydrolysis of organic compounds are provided.

One embodiment of a method of making a bacterial cellulose-metal-organic framework composite includes the steps of: preparing an aqueous fermentation medium comprising water, a cellulose-producing bacteria, a carbon source, and a nitrogen source; adding metal-organic framework particles to the aqueous fermentation medium and fermenting the aqueous fermentation medium to form a hydrogel comprising the metal-organic framework particles embedded in a bacterial cellulose nanofiber network; and converting the bacterial cellulose hydrogel having the metal-organic framework particles dispersed therein into an aerogel comprising the metal-organic framework particles embedded in a bacterial cellulose nanofiber network.

One embodiment of a bacterial cellulose-metal-organic framework composite includes: an aerogel comprising metal-organic framework particles embedded in a bacterial cellulose nanofiber network; and, optionally, a polymeric base on the aerogel, in the aerogel, or on and in the aerogel.

One embodiment of a method of disabling a toxic agent comprising an organophosphate compound having a hydrolysable bond includes the steps of: exposing the organophosphate compound to a composite in the presence of water, wherein the composite includes: an aerogel comprising metal-organic framework particles that are catalytic for the hydrolysis of the organophosphate compound embedded in a bacterial cellulose nanofiber network; and a polymeric base on the aerogel, in the aerogel, or on and in the aerogel, whereby the metal-organic framework particles catalyze the hydrolysis of the hydrolysable bond.

### BRIEF DESCRIPTION OF THE DRAWINGS

Illustrative embodiments of the invention will hereafter be described with reference to the accompanying drawings, wherein like numerals denote like elements.

FIG. 1 shows a schematic illustration of the hydrolysis of the nerve agent soman using a bacterial cellulose-metal-organic framework composite.

FIG. 2 shows a schematic illustration of biosynthesis strategy for a MOF/bacterial cellulose (BC) composite. This composite has dimensions of 6 cm×6 cm×0.3 cm.

FIGS. 3A-3F show characterizations of a MOF-808/BC composite. FIG. 3A shows PXRD patterns: FIG. 3B shows nitrogen sorption isotherms; FIGS. 3C-3F show SEM images of (FIG. 3C) BC, (FIG. 3D) MOF-808/BC-1, (FIG. 3E) MOF-808/BC-2, and (FIG. 3F) MOF-808/BC-3. Scale bars are 4 μm.

FIG. 4 shows PXRD patterns of an activated UiO-66-NH₂/BC composite.

FIG. 5 shows a N₂ sorption isotherm of an activated UiO-66-NH₂/BC composite.

FIG. 6 shows a SEM image of an activated UiO-66-NH₂/BC composite from the biosynthesis. Scale bar: 5 μm.

FIG. 7 shows solvent resistance performance of an MOF-808/BC composite. N₂ sorption isotherms of the MOF-808/BC-3 after 24 h treatment in different solvents are shown.

FIG. 8 shows PXRD patterns of MOF-808/BC-3 after 24 h treatment in different solvents under high temperature with stirring.

FIG. 9 shows SEM images of MOF-808/BC-3 after 24 h treatment in different solvents. Scale bars: 5 μm.

FIG. 10A shows a hydrolysis reaction of DMNP by a MOF-808 based catalyst. FIG. 10B shows a liquid phase hydrolysis profile of DMNP conversion with a MOF-808/BC composite in 0.4 M N-ethylmorpholine solution. Conditions: 1.5 μmol MOF-808 in composite (corresponding to 6 mol % catalyst) and 25 μmol DMNP used in catalysis reaction. FIG. 10C shows DMNP solution uptake percentage and conversion using different composites.

FIGS. 11A-11B show SEM images of MOF-808/PET-1. Scale bar: 150 μm (FIG. 11A) and 30 μm (FIG. 11B).

FIG. 12 shows a PXRD pattern of MOF-808/PET-1.

FIG. 13 shows a N₂ sorption isotherm of MOF-808/PET-1.

FIG. 14A shows the structure of crosslinked BPEI as the base for catalyst regeneration in BPEIH. FIG. 14B shows a solid-state hydrolysis profile of DMNP conversion with MOF-808/BC-1/BPEIH composite at 50% RH. Conditions: 1.5 μmol MOF-808 in composite (corresponding to 6 mol % catalyst) used in these two cases, 25 μmol DMNP. FIG. 14C shows a stability test of MOF-808/BC-1/BPEIH against different challenges, reaction time 10 min.

FIG. 15 shows PXRD patterns of a MOF-808/BC-1/BPEIH composite.

FIG. 16 shows a GD permeation test through MOF/fiber materials.

### DETAILED DESCRIPTION

MOF/BC composites, methods of making the composites, and methods of using the composites in the hydrolysis of organic compounds are provided. The composites are aerogels comprising metal-organic framework particles embedded in a bacterial cellulose nanofiber network.

The composites are fabricated using a microbial synthesis strategy in which BC nanofiber is biosynthesized using a cellulose-producing bacteria in a fermentation medium in which MOF particles are dispersed. Fermentation of the medium produces a BC nanofiber hydrogel comprising a bacterial cellulose nanofiber network in which the MOF particles are embedded in voids between the cellulose nanofibers. The removal of water and organics from the hydrogel and the pores of the MOF nanoparticles converts the hydrogel into an aerogel composed of the bacterial cellulose nanofiber network with the MOF particles embedded therein.

Because the BC is grown in situ in the presence of pre-formed MOFs in an aqueous fermentation medium, the MOF/BC composites can be produced at low temperatures and/or in the absence of organic solvents. As such, the present methods are superior to methods in which MOFs are formed from precursors in situ in the presence of BC because the in situ formation of the MOFs is typically carried out at high temperatures, generally exceeding 100° C., using organic solvents such as N,N-dimethylformamide (DMF), N,N-dimethylacetamide (DMAC), and/or dimethyl sulfoxide (DMSO), which may be trapped in composites and pose a high risk to human health if they are used as protective gear like a mask filter. Moreover, the production of MOFs in situ during the formation of a MOF/BC composite using high temperatures and highly flammable organic solvents is challenging in industrial settings.

In addition, pre-forming the MOFs obviates the exposure of the fermentation bacteria and cellulose to the MOF synthesis chemicals, enables improved control over the type, size, and shape of the MOFs that are incorporated into the composites, allows for mixtures of two or more different types of MOF to be incorporated into the composite, and increases the loading of the MOFs in the composite, relative to the loadings are achieved by synthesis strategies in which the MOFs are formed in situ in the presence of cellulose.

The composites are characterized by a hierarchically porous structure, high MOF loadings, solvent resistance, and processability. By way of illustration, composites having an MOF loading of at least 60 weight percent (wt. %), at least 70 wt. %, and at least 80 wt. %, including MOF loadings in the range from 60 wt. % to 90 wt. %, based on the combined weight of the BC nanofiber network and the MOF particles, can be synthesized.

The use of BC fibers as the matrix material in the composites is advantageous because the fibers form a network with a hierarchical pore structure, mechanical and thermal stability, and excellent biosafety. BC is a cellulose that is produced by the bio-polymerization of a carbon source, such as a sugar or an alcohol, during fermentation using a variety of microorganisms. The fermentations may be carried out without the use of hazardous or environmentally unfriendly solvents at low temperatures, including temperatures at or near room temperature. For example, fermentation temperatures of less than 60° C. of less than 40° C., of less than 30° C., of less than 25° C., and in the range from 22° C. to 30° C. can be used. However, suitable fermentation temperatures are not limited to temperatures in this range.

The BC fibers are produced in an aqueous fermentation medium that includes water, a cellulose-producing bacterium, a carbon source, and a nitrogen source. Additional components, such as mineral sources, typically found in BC fermentation media can also be included. Examples of cellulose-producing bacteria include Gluconacetobacter (e.g., Gluconacetobacter xylinus), which also known as Acetobacter, Agrobacterium, Aerobacter, Achromobacter, Azotobacter, Rhizobium, Sarcina, and Salmonella. The fermentation medium is an aqueous medium in which water is the majority solvent, and is preferably the only solvent. While it is possible to include small amounts of non-water solvents in the fermentation mediums, such non-water solvents should be present at a low concentration relative to water; for example, if organic solvent is present in the aqueous fermentation medium, said medium desirably has a water:organic solvent ratio less than 10:1 (v/v), more desirably less than 50:1, and still more desirably less than 100:1. In particular, polar aprotic solvents, such as DMF, DMAc, and DMSO, and/or alcohols, such as ethanol, may be excluded from the fermentation medium.

Various carbon and nitrogen sources may be used in the fermentation media, and the selection of the best candidates may depend on the particular bacteria being used. Illustrative examples of carbon sources include sugars and sugar alcohols, such as fructose, glucose, glycerol, inositol, mannitol, sorbitol, succinic acid, sucrose, tryptose, and combinations of two or more thereof. Illustrative examples of nitrogen sources include yeast extract, peptone, beef extract, malt extract, casein hydrolysate, and combinations of two or more thereof. Illustrative additives that may, optionally, be included in the fermentation media include polyethylene glycol (PEG), agar, cellulose, chitin, gelatin, lignin, pectin, xylan, and combinations of two or more thereof.

Illustrative concentrations for the carbon source and the nitrogen source in the fermentation media are from 3% (v/v) to 15% (v/v), including from 8% (v/v) to 12% (v/v), and from 0.25% (v/v) to 5% (v/v), including from 1% to 3% (v/v), respectively. However, concentrations outside of these ranges can be used.

Once the aqueous fermentation medium (also referred to as a culture medium) is formed. MOF particles are added to the medium. In the presence of the MOF particles, fermentation of the medium results in the production of a hydrogel comprising the metal-organic framework particles embedded in a bacterial cellulose nanofiber network. Once formed, the hydrogel may be washed with water to lyse the bacteria and remove fermentation media.

The hydrogel is then converted into an aerogel by removing the liquid from the pores of the hydrogel. This can be accomplished, for example, by supercritical CO₂ drying of the hydrogel to remove residual fermentation medium and water from the pores. The supercritical drying can be rendered more efficient if it is preceded by a solvent exchange to exchange the existing pore-filling liquids with a solvent having a lower critical temperature and lower critical pressure, such as ethanol. Notably, the conversion from a hydrogel to an aerogel can be carried out without sacrificing the porous structure of the gel.

A variety of different MOFs can be used in the methods. The MOF particles are composed on MOFs, which are hybrid, crystalline, porous compounds made from metal-ligand networks that include inorganic nodes connected by coordination bonds to multitopic organic linkers. The inorganic nodes (also referred to as vertices) in the framework include metal ions or clusters. (By convention, carboxylates or other linker terminal groups or atoms are often represented as components of the nodes.)

In some embodiments of the methods and the composites formed therefrom, the MOF particles are composed of MOFs having Lewis acidic sites that are catalytically active for the hydrolysis of one or more organic compounds, such as organophosphonate compounds, including such compounds that are used as chemical warfare agents. Composites incorporating such MOF particles can be incorporated into a variety of protective wearable articles, such as filters in face masks, gloves, and body suits. It should be understood, however, that the composites described herein are not limited to those in which the MOFs are catalytically active for the hydrolysis of organic compounds; other types of MOFs can be used.

Examples of MOFs that can be used for organic nerve agent hydrolysis include hexanuclear zirconium cluster-based MOFs (Zr-MOFs) with diverse pore geometries, pore sizes, and catalytically-active Zr₆ nodes. Descriptions of such MOFs can be found in Bai, Y. et al., Chem. Soc. Rev. 45, 2327-2367, (2016); Cavka, J. H. et al., J. Am. Chem. Soc. 130, 13850-13851, (2008); and Chen, Z. et al., Coord. Chem. Rev. 386, 32-49. (2019). Specific examples of suitable MOFs include, but are not limited to, those designated in the literature as MOF-808, UiO-66, UiO-66-NH₂, NU-1000, NU-901, NU-1601, and NU-1602, as well as other UiO (Universitetet I Oslo) and NU (Northwestern University) series MOFs. Information about the structures of these Zr-MOFs is provided in Table 1 and in Example 1.

TABLE 1

| | Net | | Linker | Cluster | |
|---|---|---|---|---|---|
| | (rcsr | | connectivity | connectivity | |
| MOF | code) | Connectivity | and shape | and shape | Ref. |
| NU- | alb | 6,12-c | 6-c, trigonal | 12-c, hexagonal | 1 |
| 1601 | | | prism | prism | |
| | | | | (disordered) | |
| NU- | alb | 6,12-c | 6-c, trigonal | 12-c, hexagonal | 1 |
| 1602 | | | prism | prism | |
| | | | | (disordered) | |
| UiO- | fcu | 12-c | 2-c, linear | 12-c, | 2 |
| 66 | | | | cuboctahedron | |
| NU- | scu | 4,8-c | 4-c, rectangle | 8-c, cube | 3 |
| 901 | | | | | |
| NU- | csq | 4,8-c | 4-c, rectangle | 8-c, cube | 4 |
| 1000 | | | | | |
| MOF- | spn | 3,6-c | 3-c, triangular | 6-c, trigonal | 5 |
| 808 | | | | antiprism | |

The references recited in Table 1 are as follows: Ref. 1-Chen, Zhijie, et al., Journal of the American Chemical Society 141.31 (2019): 12229-12235; Ref. 2-Cavka, J. H. et al., J. Am. Chem. Soc. 2008, 130, 13850-13851; Ref. 3-Kung, C.-W. et al., Chem. Mater. 2013, 25, 5012-5017; Ref. 4-Mondloch, J. E. et al., J. Am. Chem. Soc. 2013, 135, 10294-10297; and Ref. 5-Peterson, G. W. et al., J. Porous Mater. 2014, 21, 121-126; Ref. 4-Feng, D. et al., J. Am. Chem. Soc. 2014, 136, 17714-17717; Ref. 5-Lyu, J. et al., Chem. Sci. 2019, 10, 1186-1192.

It should be understood that, although the preceding description focuses on zirconium MOFs, other isostructural MOFs having the same network topologies can be used. These isostructural MOFs differ from the Zr MOFs described herein with respect to the nature of the metal in the inorganic nodes. For example, isostructural hafnium MOFs, cerium MOFs, and thorium MOFs can be synthesized using hafnium salts, cerium salts, and thorium salts, respectively. In should be further understood that the specific MOFs listed above and their isostructural counterparts are not presented as an exhaustive list. As the methods described herein use pre-formed MOFs and, therefore, separate the MOF synthesis chemistry from the cellulose fermentation step, virtually any pre-formed MOF may be used. These include non-zirconium MOFs, such as, but not limited to, iron-based MOFs and copper-based MOFs, and zirconium-based MOFs other than those belonging to the UiO and NU series. Other, non-limiting examples of MOFs include MOFs in the MIL (Materials of Lavoisier) series, such as MIL-53, MOFs of the MIP (Materials of the Institute of Porous Materials of Paris) series, such as MIP-202 and MIP-206, and MOFs of the HKUST (Hong Kong University of Science and Technology) series. While composites that include one or more of the MOFs listed herein are contemplated, composites that exclude one or more of the specific MOFs or that exclude a particular series of MOF listed herein are also contemplated.

Optionally, in order to increase the effectiveness of the composites at hydrolyzing bonds in organic molecules, polymeric bases can be introduced into the composites by, for example, coating the composites with the polymeric bases and/or integrating the polymeric bases into the pores of the composite. The polymeric bases are organic polymers that have Lewis acidic sites. Polyethylenimine (PEI), including linear PEI, branched PEI, and PEI dendrimers, can be used as the polymeric base. Other suitable organic polymeric bases include poly(allylamine), poly(N-methylvinylamine), chitosan, and polydopamine. The polymeric bases can be homopolymers or copolymers. The polymeric bases may be non-crosslinked or crosslinked. In some embodiments of the composites, the polymeric bases are crosslinked and form a hydrogel that is incorporated into the open pores of the aerogel of the composites. The polymeric base hydrogels form a three-dimensional network of crosslinked polymer chains in which water fills voids between the polymer chains. The polymeric bases can be incorporated onto and/or into the composites by, for example, exposing the composite to a solution containing the polymeric base, whereby the polymeric base adsorbs onto the composite, following by drying. For embodiments of the composites in which the polymeric base is a hydrogel, the polymeric base can be incorporated into the composites by exposing the composite to a solution containing the polymeric base and a crosslinking agent and incubating the composite at a temperature and for a time sufficient for a polymeric base hydrogel to form.

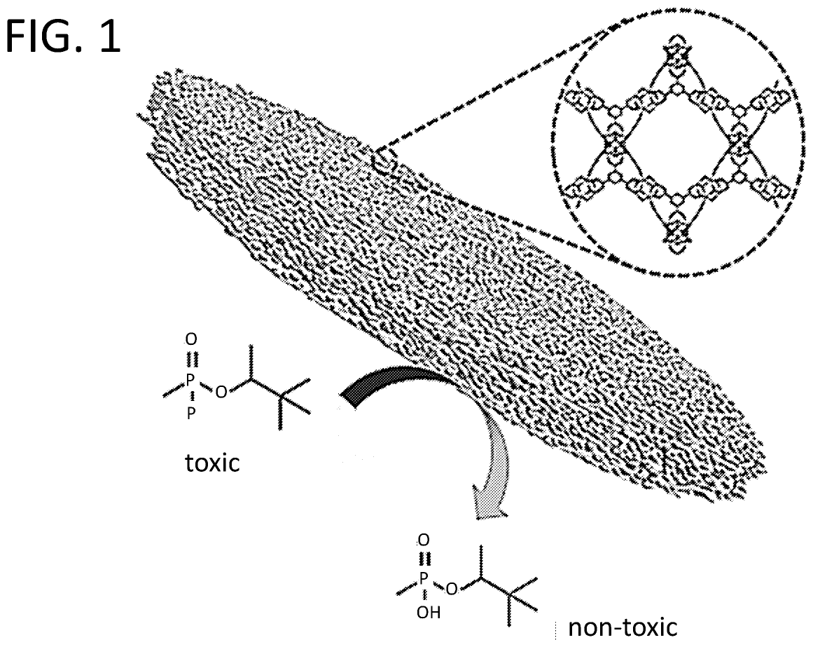

The composites containing MOF particles that are catalytically active for hydrolysis reactions can be exposed to an environment containing water and an organic compound having a hydrolysable bond, such as an organophosphonate molecule. The water in the environment may be in liquid form or in vapor form, as in a humid environment. Lewis acidic sites on the MOFs, such as Zr—OH—Zr sites, catalyze the hydrolysis of the organic compounds, thereby detoxifying them. The polymeric bases then regenerate the Lewis acidic sites on the MOFs during the catalysis. This process is illustrated schematically in FIG. 1, using the compound soman as a representative organophosphate nerve agent. Using the composites described herein, organophosphates, including organophosphonates, can be converted into their hydrolyzed forms with a conversion percentage, based on moles, of 60% or higher, 70% or higher, or 90% or higher. For example, conversions in the range from 70% to 100% can be achieved.

The toxic agents that can be disabled using the composites include chemical agents that have at least one hydrolysable bond and that produce a harmful physiological reaction in a human or other animal. Examples of nerve agents that can be hydrolyzed include G and V series agents such as soman, sarin, tabun, cyclosarin and [2-(Diisopropylamino)ethyl]-O-ethyl methylphosphonothioate (VX).

### Example

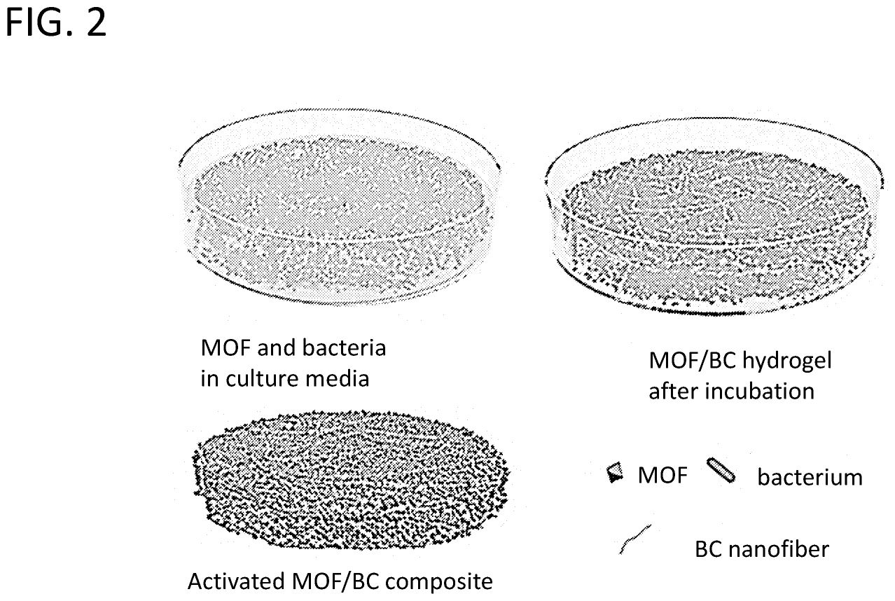

This example describes an environmentally benign biosynthesis method for the facile introduction of MOF nanoparticles into a cellulose nanofiber network during its fermentation, thereby forming a functional MOF composite material (FIG. 2). Gluconacetobacter xylinus (ATCC 23770) was employed as the strain to generate a bacterial cellulose network to support the MOF nanoparticles. After a culture of bacteria was in culture media with MOF nanoparticles for 5 days, a solidified MOF/BC hydrogel was obtained, which could be activated by super critical CO₂. The resulting composite had low-density, high MOF mass loading, hierarchical porosity, and excellent solvent resistance behavior. The well dispersed MOF nanoparticles in the cellulose nanofiber composite showed improved catalytic detoxification and uptake of toxic organophosphorus nerve agents.

### Results and Discussion

Nanoparticles of MOF-808, a MOF comprised of Zr₆ nodes and benzene tricarboxylate organic linkers, was used as a high-performing nerve agent detoxifying catalyst. The MOF-808 nanoparticles (50 nm) were mixed with Gluconacetobacter xylinus in culture media containing sucrose, yeast extract, and peptone, to get a stable slurry. After room temperature fermentation for 5 days in the optimized culture media containing 1% yeast extract, 1% peptone, and 10% sucrose, the MOF functionalized cellulose nanofiber hydrogels were obtained. After washing with water and exchanging with ethanol, the gels were then activated using super critical CO₂ to yield the porous MOF-808/BC monolithic composites.

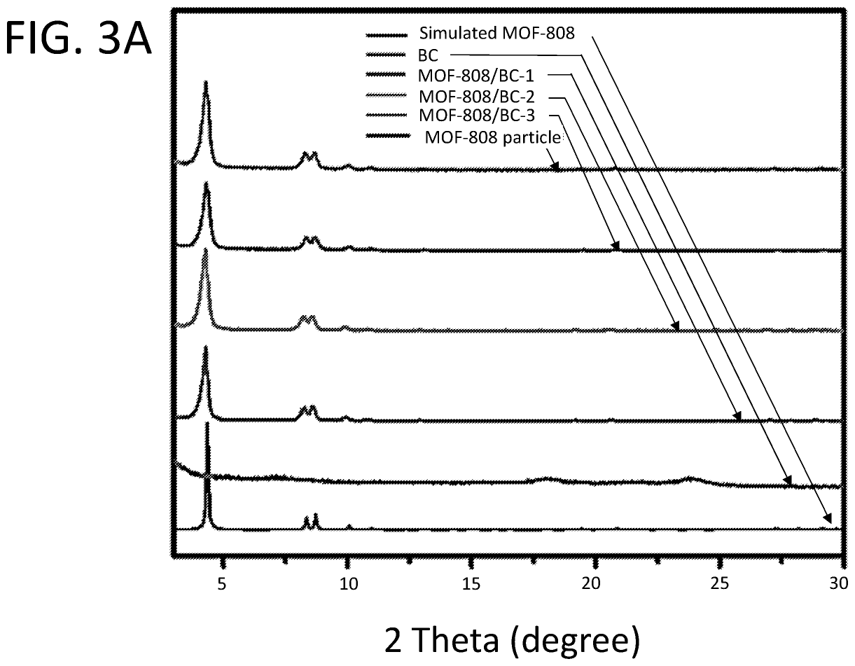

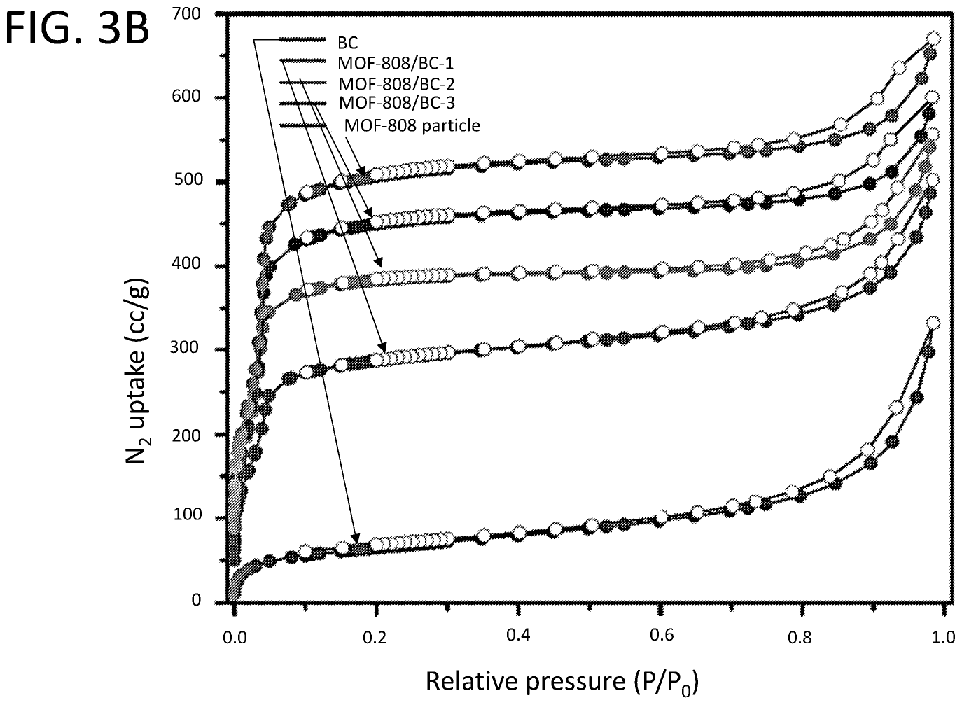

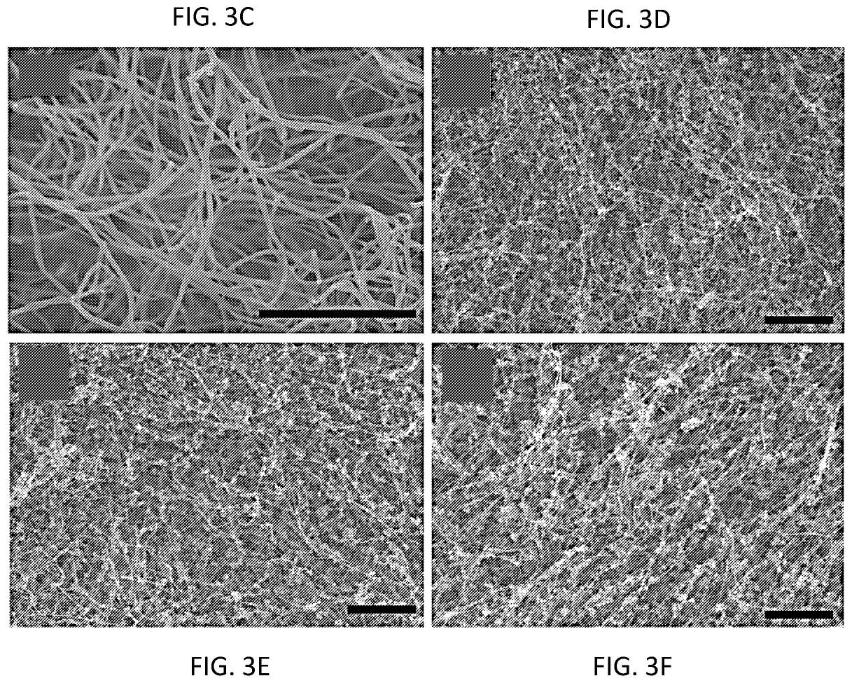

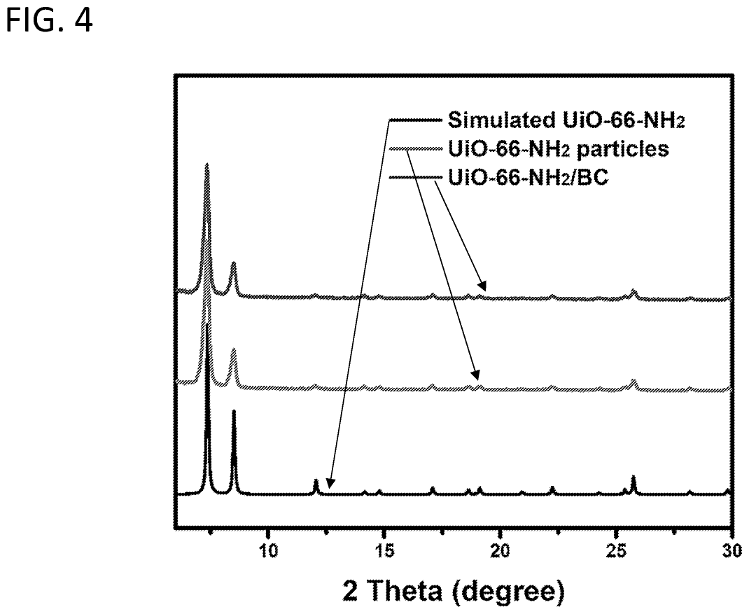

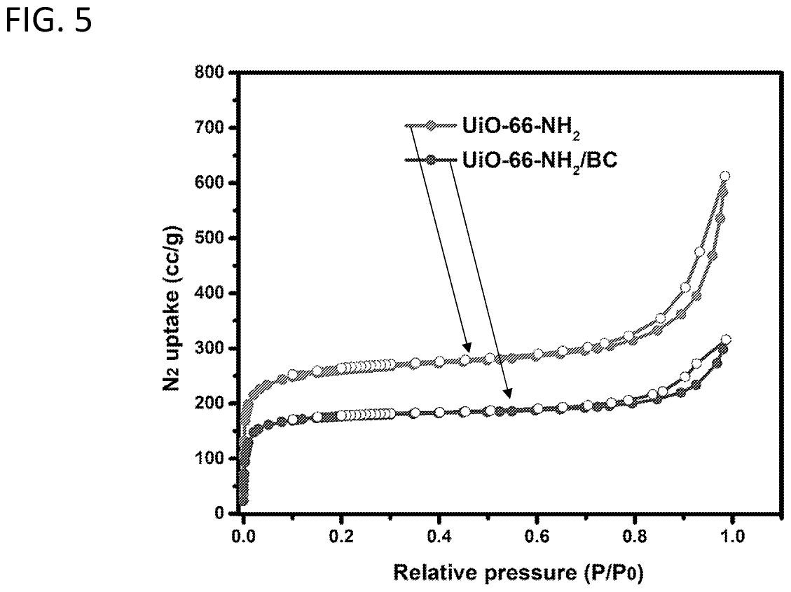

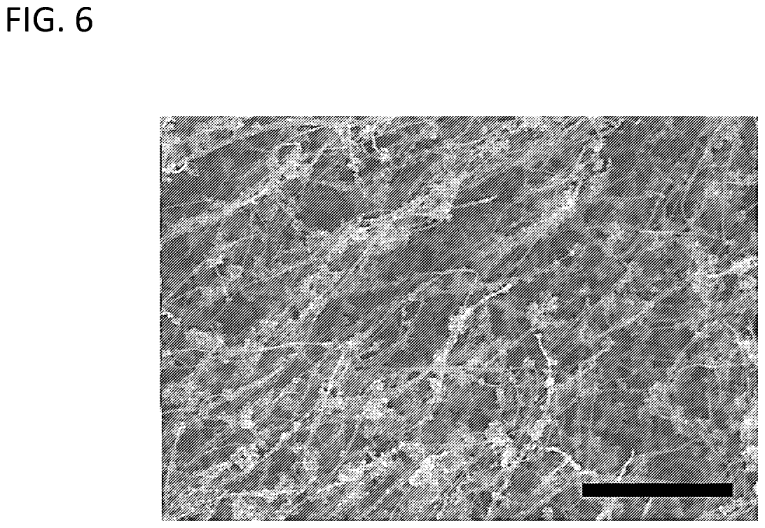

The activated MOF-808/BC composites, which originated from the culture media with 40 mg/mL, 80 mg/mL, and 160 mg/mL MOF-808, were named MOF-808/BC-1, MOF-808/BC-2, and MOF-808/BC-3. Based on inductively coupled plasma-optical emission spectrometry (ICP-OES), the MOF mass loadings in the composite were 60%, 75%, and 90%, respectively, for MOF-808/BC-1, MOF-808/BC-2, and MOF-808/BC-3, indicating the mass loading can tuned by adjusting the MOF concentration in the culture media. The crystalline nature of the composites was verified by powder X-ray diffraction (PXRD) measurements, and the newly introduced patterns matched well with the simulated MOF-808 (FIG. 3A) pattern. The porosity of the composites was probed by N₂ uptake at 77 K. As the MOF loading increased, the Brunauer-Emmett-Teller (BET) surface areas (S_{BET}) of the composites improved from 120 m²/g (pure bacterial cellulose) to 1100 m²/g (MOF-808/BC-1), 1420 m²/g (MOF-808/BC-2), and 1620 m²/g (MOF-808/BC-3) (FIG. 3B; Table 2). The normalized BET surface of MOF-808 component in the composite ranged from 1800 m²/g to 1890 m²/g (Table 2), in agreement with that of the pristine MOF-808 nanoparticle (1950 m²/g). (Mondloch, J. E. et al., Nat. Mater. 2015, 14, 512.) Importantly, scanning electron microscope (SEM) images captured the macroporosity of the composite and good spatial distribution of the MOF nanoparticles throughout the hierarchical 3D network (FIGS. 3C-3F). The densities of MOF-808/BC-1, MOF-808/BC-2, and MOF-808/BC-3 composite were 65 mg/cm³, 120 mg/cm³, and 190 mg/cm³, respectively (Table 1). Additionally, the biosynthesis method was extended to synthesize another MOF-BC composite, instead incorporating UiO-66-NH₂, a Zr₆-based MOF with 2-aminoterephthalic acid as the linker, into the bacterial nanocellulose framework. The crystallinity and microporosity and hierarchical morphology of the UiQ-66-NH₂/BC composite were verified through PXRD, N₂ sorption, and SEM (FIGS. 4-6).

TABLE 2
Physical property summary of the MOF-808/BC composites.

| | MOF | | | | | |
|---|---|---|---|---|---|---|
| | in | MOF | | | Normalized | |
| | culture | load- | | Micro- | S_{BET} of | |
| | media | ing | S_{BET} | porosity | MOF-808 | Density |
| Materials | (mg/g) | (%) | (m²/g) | (%) | (m²/g) | (mg/cm³) |
| MOF- | 40 | 60 | 1100 | 90 | 1830 | 65 |
| 808/BC-1 | | | | | | |
| MOF- | 80 | 75 | 1420 | 91 | 1890 | 120 |
| 808/BC-2 | | | | | | |
| MOF- | 160 | 90 | 1620 | 95 | 1800 | 190 |
| 808/BC-3 | | | | | | |

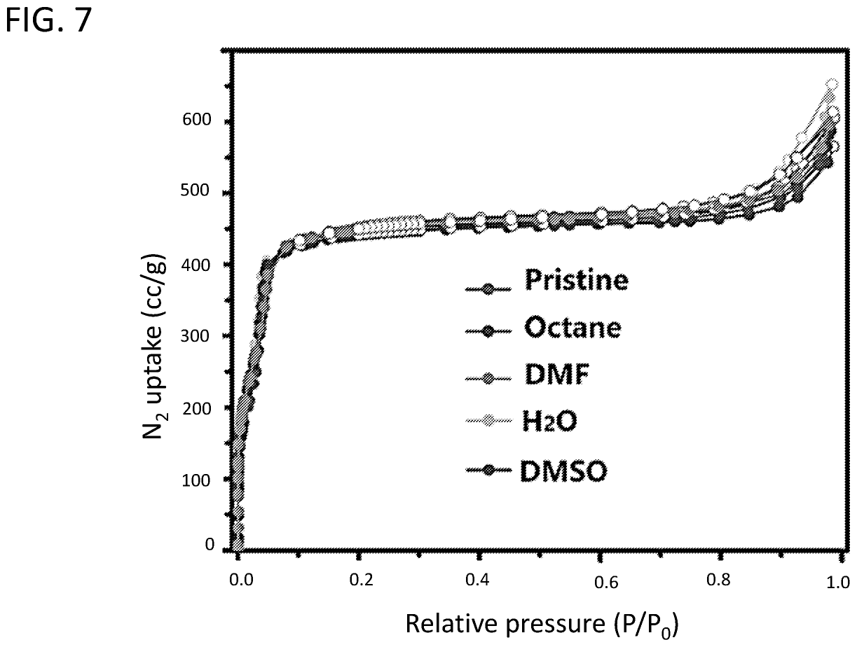

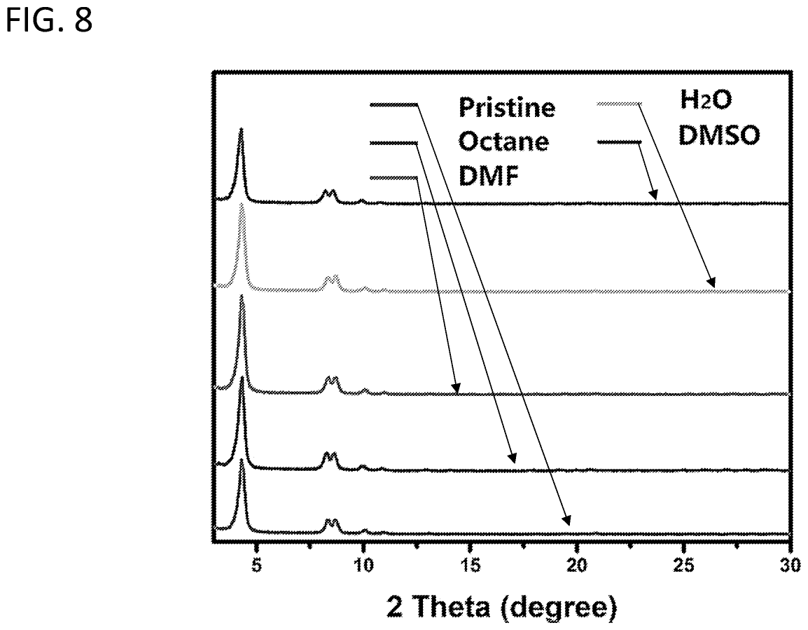

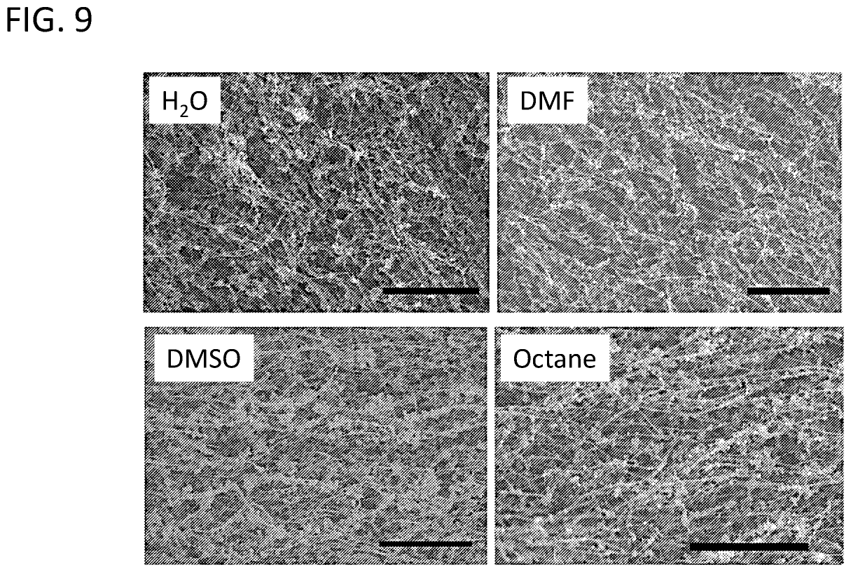

High solvent resistance performance of the functional MOF components in a composite is essential for some practical applications, as leaching of the functional nanoparticles from the composites could reduce their working capacities and lifetimes. The solvent resistance property of MOF-808/BC-3 was firstly tested through stirring in water (100° C.), the organic polar solvents, DMF (120° C.) and DMSO (120° C.), as well as nonpolar solvent octane (110° C.) at 200 rpm for 24 h. These composites maintained their shape, and there was no obvious leaching of particles into the solvent after solvent treatment under heating. Quantitative measurement by ICP-OES also confirmed the unchanged MOF loading in the composites, and N₂ uptake study showed that the MOF-808/BC composites maintained the porosity after solvent treatment (FIG. 7). Moreover, the intact crystallinity and hierarchical morphology after the solvent resistance test were verified by PXRD and SEM tests, respectively (FIGS. 8-9). The bacterial cellulose contains microfibrils with abundant hydrogen bonds between the glucose subunits of the adjacent cellulose macromolecules, which can help stabilize the bulk fiber in a range of solvents. Thus, the excellent stability of the MOF/BC composite in various solvents further expands the compatibility to these solvents and enables further applications. Moreover, the aqueous biosynthesis of the MOF/BC composites avoids the use of organic solvents with a high boiling point and improves the use of the composites as a health-care material or protective gear.

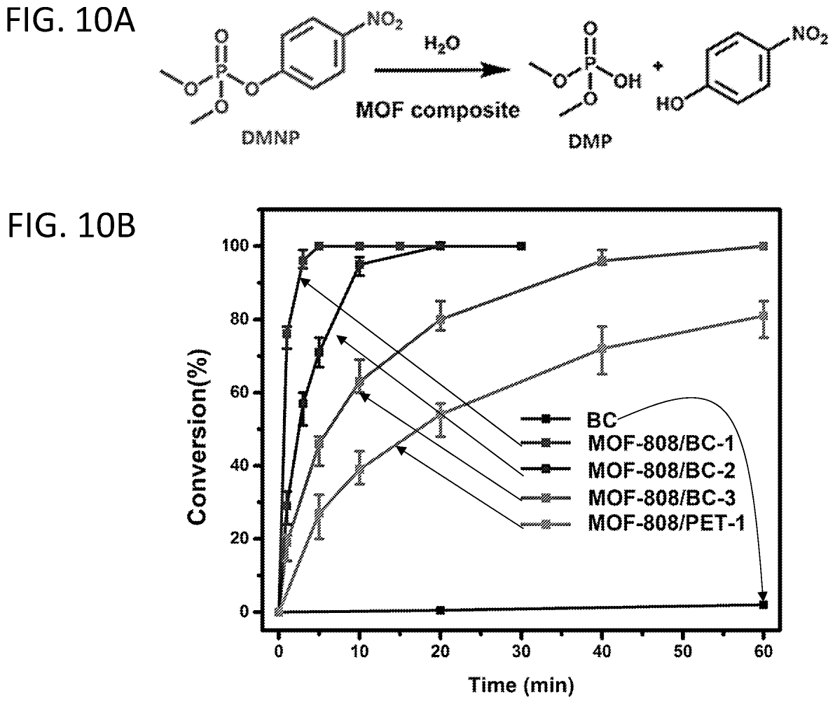

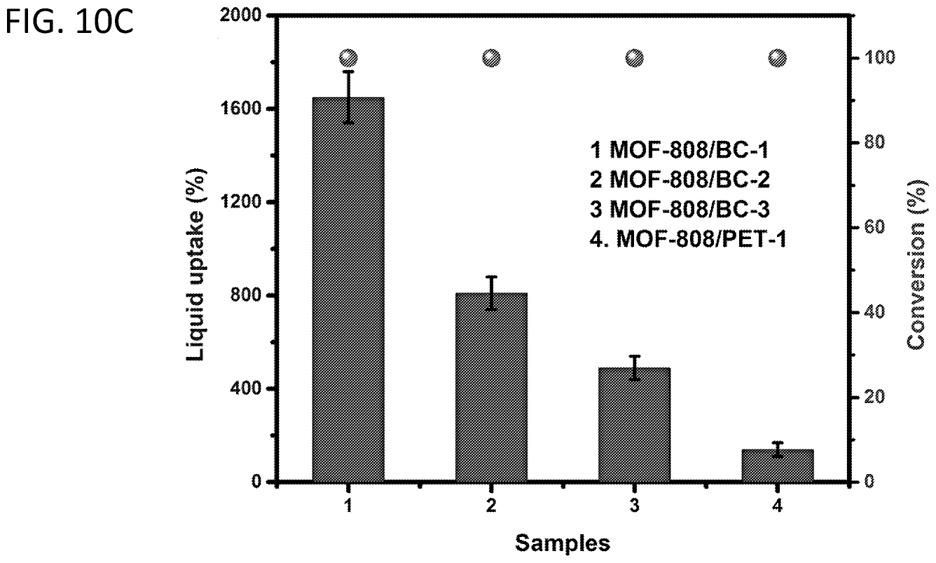

Given its 3-D hierarchical structure, good stability, and high loading of a MOF-based chemical warfare agent (CWA) detoxification catalyst, the MOF/BC composite was explored for its potential as a dual-functional absorbing and detoxifying material. A liquid phase catalytic hydrolysis reaction of a nerve agent simulant, dimethyl-4-nitrophenyl phosphate (DMNP), in 0.4 M N-ethylmorpholine solution was used to probe the catalytic activity of the MOF-808/BC composite, and the conversion of DMNP to nontoxic dimethyl phosphate (DMP) was monitored by ³¹P nuclear magnetic resonance (NMR) as shown in FIG. 10A.

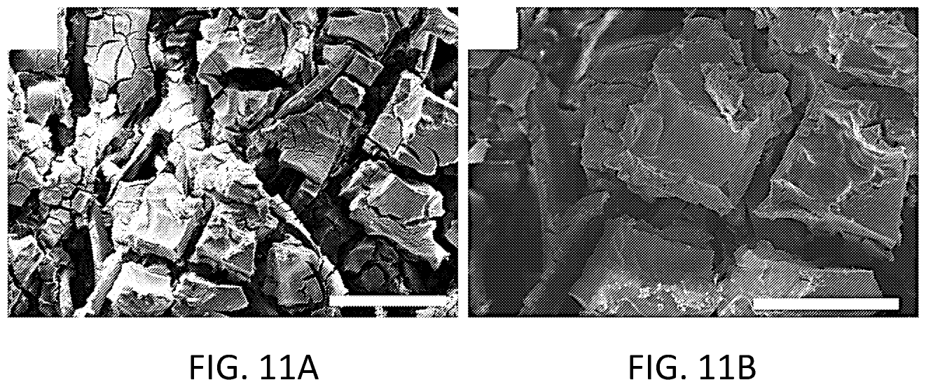

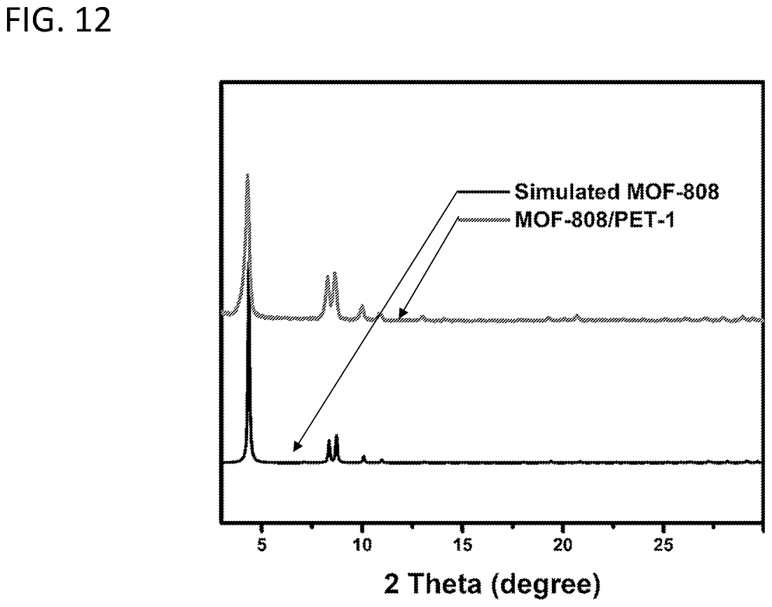

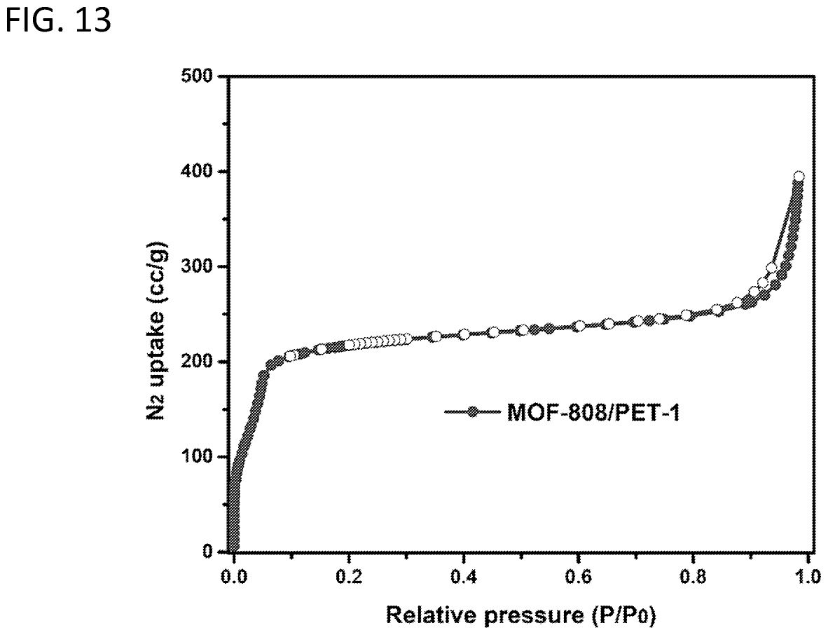

As shown in FIG. 10B, the MOF-808/BC-1 composite achieved conversions of 76% and ˜100% after 1 min and 5 min of reaction time, which indicated a half-life within 1 min, almost the same as free MOF-808 nanoparticles. With the increased MOF loading, MOF-808/BC-2 and MOF-808/BC-3 composites showed a half-life of about 3 min and 7 min, respectively. The DMNP was nearly selectively hydrolyzed after approximately 5 min for MOF-808/BC-1, 20 min for MOF-808/BC-2, and 60 min for MOF-808/BC-3 (FIG. 10B), while the BC itself did not show obvious conversion after 60 min. As the same amount of MOF-808 was used in all catalytic tests (6 mol %, normalized based on Zr₆ node amount), the composite with lower MOF loading had larger volumetric size and increased external surface for the better diffusion of the agent to the catalyst, favoring the improvement of the catalytic activity. In another comparison study, a commercial polyethylene terephthalate textile fiber coated with a MOF-808 layer (50 wt %, termed as MOF-808/PET-1. FIGS. 11-13), showed much slower kinetics in the catalytic reaction with a half-life of 20 min, indicating MOF-808/BC-1 showed a 20-fold increase in catalytic activity with the similar MOF mass loading. To achieve a higher MOF loading on the PET commercial textile, the PET fiber surface was covered in a dense MOF coating on the fiber surface, which limited the accessible pathways for chemical diffusion (FIG. 11). In contrast, the superior spatial dispersion present in the MOF-808/BC composite enabled a more accessible MOF catalyst in the hierarchical network for enhanced reagent diffusion and subsequently a faster reaction rate. Therefore, the MOF-808/BC composite maintains highly accessible catalytic sites even at high MOF loadings, which is not easily obtainable by formerly reported material.

Given the high porosity composite and high loading of catalyst in the MOF/BC aerogel, its potential for simultaneously absorbing and detoxifying DMNP from water (FIG. 10C) was explored. MOF-808/BC-1 with the lowest density showed a liquid uptake of 1650 wt %, while MOF-808/BC-2 and MOF-808/BC-3 showed a lower uptake of 810 wt % and 490 wt %, respectively. The higher liquid uptake of MOF-808/BC-1 is attributed to its lower density. Interestingly. MOF-808/PET-1, with fewer macropores, showed a liquid uptake of only 140%, which is 10 times lower than that of MOF-808/BC-1, though with a similar MOF mass loading. After stopping the catalytic reaction by filtering the liquid from the composite, the catalytic hydrolysis of the absorbed simulant was studied using the ³¹P NMR method. The results showed that all of the MOF-808/BC composites showed a total conversion within 2 min (FIG. 10C). In addition, the catalytic performance of the MOF-808/BC composite was fully recovered after washing and reactivation, showing good recyclability. Moreover, the sample, after aging in air for 4 months, showed intact absorption capacity and catalytic activity. Therefore, the MOF-808/BC composite possesses high catalytic efficiency, capacity, and stability. The high liquid uptake and simultaneous fast degradation makes this material a useful detoxifying absorbent against a nerve agent spill.

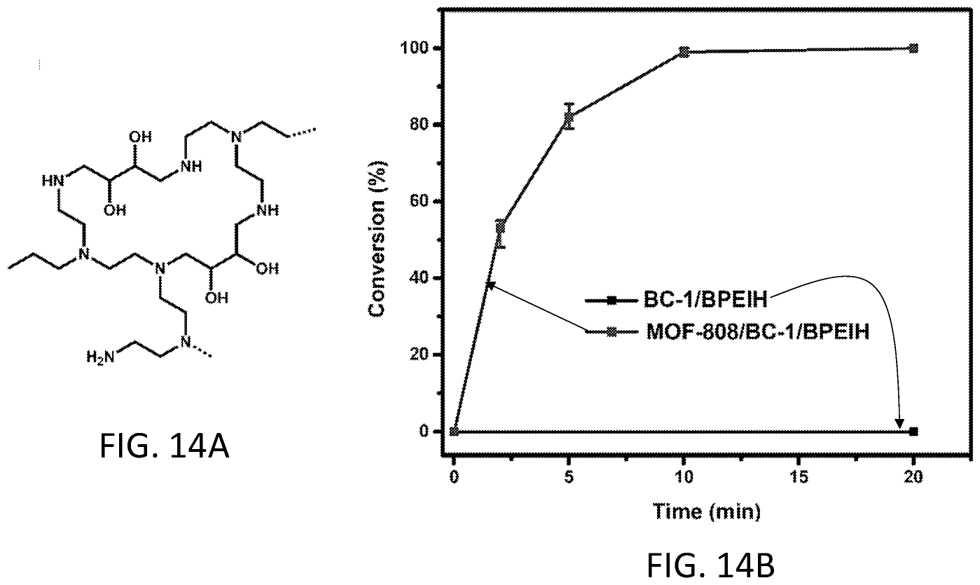

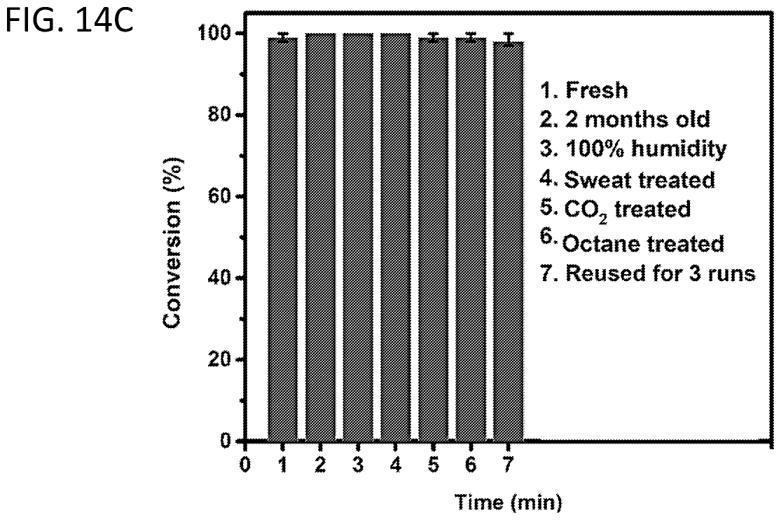

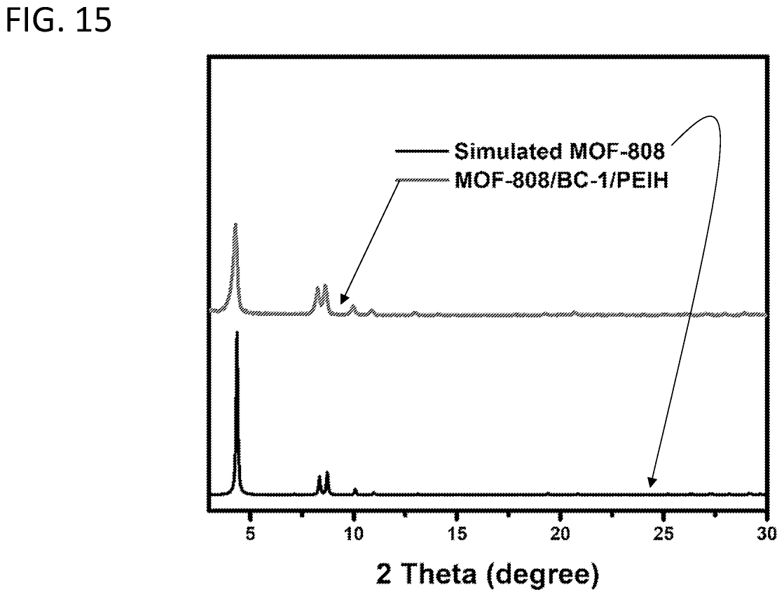

To further improve the practicality of the composite used in protective gear, a crosslinked branched polyethyleneimine hydrogel (BPEIH) was introduced to the porous MOF-808/BC-1 composite (termed as MOF-808/BC-1/BPEIH, FIG. 14A and FIG. 15). The polymeric BPEIH could supply a nonvolatile polymeric base and water source to mediate the micro-environment in composite and to boost the catalytic hydrolysis of nerve agents and their simulants. At the same time, the hierarchical structure of the composite enables agent diffusion, with increased contact with the catalyst for faster hydrolysis. Solid-state hydrolysis of DMNP was carried out to probe the catalytic performance of this MOF-808/BC-1/BPEIH by adding 4 μL simulant on the composite's surface (0.7 cm×0.7 cm×0.015 cm, with 6 mol % catalyst) directly under ambient humidity (relative humidity=50%). The extent of the hydrolysis reaction was monitored by ³¹P NMR following the digestion of the composite after a certain contact time. A respective conversion of 53% and 99% after 2 and 10 min was obtained (FIG. 14B), and these kinetic studies indicate a short initial half-life of about 2 min for the degradation of DMNP. The catalytic activity is very similar to that of the reaction in organic base solution, while the monolithic composite catalyst is practical as a mask filter against different nerve agent threats. The MOF-808/BC-1/BPEIH composite was aged for 2 months in a sealed vial and fully converted DMNP within 10 min, consistent with the fresh sample (FIG. 14C). The composite was regenerated through simple water washing, and it maintained the same conversion of DMNP (98% at 10 min). The composite also showed good tolerance against different environmental stressors. After exposure to sweat, 100% humidity, and a CO₂ or octane-rich atmosphere, the MOF-808/BC-1/BPEIH did not show any degradation in efficiency, implying a good practicality of the composite materials (FIG. 14C).

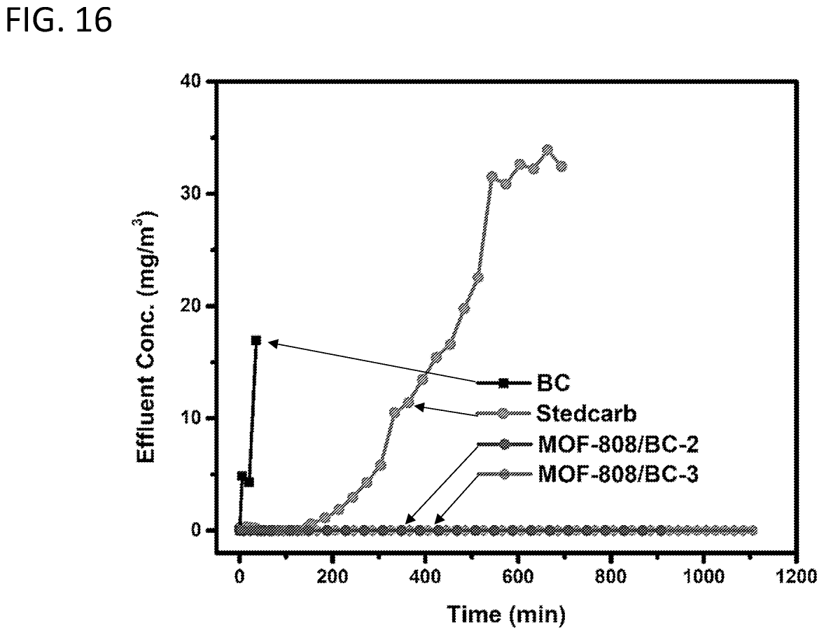

The high porosity, good accessibility, and reactivity of the MOF-808/BC composite also showed great potential for chemical warfare agent capture from contaminated air flow, which is essential for protective cloth to reduce the toxicity to humans. Ideally, high retention time of the toxic agent is desired to increase the contact barrier from the human body. A standard test (ASTM F739-12) for the permeation of toxic gases through protective clothing materials was carried out to evaluate the capture capacity and protective barrier properties of a real nerve agent, O-pinacolyl methylphosphonofluoridate (GD), through the MOF-808/BC composite. (Montoro, C. et al., J. Am. Chem. Soc. 2011, 133, 11888-11891.) The GD analyte, diluted in air with a concentration of 300 mg/m³, was fed through a piece of porous composite (1 inch in diameter) at a flow rate of 300 mL/min. The GD permeation through the composites in the exiting streams was monitored using an Agilent 6890 gas chromatograph equipped with a flame ionization detector, and the results are summarized in FIG. 16. The analyte fully permeated through the pure BC mat immediately, indicating limited capture capabilities from the BC sample without the integration of the MOF-808 component. A commercialized adsorptive carbon cloth (Stedcarb) with similar thickness showed a total protection of the GD from steam for 130 min. Amazingly, MOF-808/BC-2, and MOF-808/BC-3 demonstrated no permeation for at least 910 min and 1100 min, indicating an enhanced protection duration by at least 7- and 8.5-fold, respectively, in comparison to the carbon cloth. The dose extraction test was also conducted by adding 10 μL GD onto 10 mg composite. 83%-98% of GD was degraded into nontoxic product after 24 h, even without the assistance from liquid water and base. Thus, these MOF-808/BC composites demonstrate dual-functional protection materials for nerve agent capture and detoxification.

### CONCLUSION

In summary, a facile biosynthesis strategy for a MOF/fiber composite to incorporate a MOF component into a cellulose nanofiber substrate during microbial fermentation is reported here. The prepared flexible MOF/nanofiber composite featured hierarchical pores and tunable MOF loading, which can be used for efficient nerve agent simulant catalytic hydrolysis and toxic nerve agent capture. The biosynthesis method proposed here employed water as the media, eliminating the risk caused by toxic solvents, which could improve the environmentally benign production of MOF-based composite materials with potential applications in important fields, such as nerve agent destruction, environmental remediation, and protection gear.

### Experimental Section

### Materials

ZrOCl₂: 8H₂O (98%), benzene-1,3,5-tricarboxylic acid (BTCA, 98%), 2-aminoterephthalic acid (H₂BDC-NH₂, 99%), formic acid, and sucrose were purchased from Sigma-Aldrich. Yeast extract, peptone, and all organic solvents for bacteria culture were purchased from Fisher. PET cloth used in control study was purchased from Amazon. Gluconacetobacter xylinus (ATCC 23770) was purchased from American Type Culture Collection.

### Instrumentation

PXRD patterns of all materials were recorded at room temperature on a STOE-STADIMP powder diffractometer equipped with an asymmetric curved Germanium monochromator (Cu-Kα1 radiation, λ=1.54056 Å) at IMSERC (Integrated Molecular Structure Education and Research Center) of Northwestern University. N₂ adsorption and desorption isotherms of all materials were tested on a Micromeritics Tristar (Micromeritics, Norcross. GA) instrument at 77 K. SEM images of all coatings were taken using a Hitachi SU8030 at the EPIC facility (NUANCE Center-Northwestern University). Before SEM observation, all samples were coated with OsO₄ to ˜9 nm thickness in a Denton Desk III TSC Sputter Coater ICP-OES was tested using an iCAP™ 7600 ICP-OES Analyzer (Thermo Scientific™) over the 166-847 nm spectral range. Before SEM and N₂ sorption testing, the samples were activated using supercritical CO₂ using a Tousimis Samdri-931 critical point dryer at a bleed rate of 1.0 mL/min. For DNMP hydrolysis, ³¹P NMR spectra were collected on 400 MHz Agilent DD MR-400 at IMSERC (Integrated Molecular Structure Education and Research Center) of Northwestern University. Mass loadings of MOF-808 in the composites were calculated based on ICP-OES study using an iCAP™ 7600 ICP-OES Analyzer (Thermo Scientific™) over the 166-847 nm spectral range analysis. Before ICP-OES testing, all composites were dried in a vacuum oven overnight at 100° C. Typically, 100 mg of composite was quickly weighted and digested in 10 mL HNO₃. 100 μL of the above solution was diluted into 10 mL using Milli-Q water to test the Zr concentration using the ICP-OES method. The mass loading (ML) was calculated using the following equation: ML=(C×100×0.01 L)/(100 mg×Wzᵣ)×100%, where C is the concentration of Zr in the diluted nitric acid solution measured by ICP-OES, mg/L. 100 is the dilution factor, and 0.01 L is the volume of concentrated HNO₃ used in digestion of the fiber composite. Wzᵣ is the mass percentage in MOFs: 35.3% for MOF-808 and 32.8% for UiO-66-NH₂.

### Synthesis of MOF-808 Nanoparticles

MOF-808 nanoparticles were synthesized according to a published procedure with slight modifications. (Ma, K. et al., Chem Catalysis. 2021, 1, 1-13.) Benzenetricarboxylic acid (6.3 g, 30 mmol) and ZrOCl₂·8H₂O (19.5 g, 60 mmol) were dissolved in a mixture of 450 ml DMF and 450 ml formic acid and heated at 120° C. for 48 h in a preheated oven. The product was collected by centrifugation and washed with ethanol (×3) and DI water (×3). The wet MOF-808 slurry in water was used directly as the stock for biosynthesis, as the dried MOF-808 nanoparticles were not able to be redispersed well again.

### Synthesis of UiO-66-NH₂ Nanoparticles

UiO-66-NH₂ nanoparticles were prepared according to a reported procedure with slight modifications. (Ma, K. et al., 2021.) ZrCl₄ (5 g, 21.6 mmol) and 2-aminoterephthalic acid (5.34 g, 30 mmol) were dissolved in a mixture of 150 ml of DMF and 20 mL of concentrated HCl (12 M) and heated at 120° C. for 18 h in a preheated oven. The product was collected by centrifugation and washed with ethanol (×3) and deionized (DI) water (×3). The wet UiO-66-NH₂ slurry in water was used directly as the stock for biosynthesis.

### Preparation of the Bacteria Suspension

Gluconacetobacter xylinus (ATCC 23770) was used as the strain for MOF/bacterial cellulose nanofiber composites' biosynthesis. In a typical culture media, a water solution containing 1% yeast extract, 1% peptone, and 10% sucrose was prepared. The culture media was sterilized at 115° C. for 30 min and cooled to room temperature before use. Gluconacetobacter xylinus (ATCC 23770) was pre-cultured in 500 mL fermentation media in an unsealed 1-L Erlenmeyer flask at room temperature under stirring (500 rpm) overnight, and a slight amount of fibrous solid was removed with a tweezer.

### Preparation of MOF-808/BC Hydrogel Composites

The MOF-808 nanoparticle slurry was vortexed in a 50 mL centrifuge tube to get a homogeneous mixture, and the concentration of MOF-808 in the slurry was estimated by weighing the MOF in 1 mL of slurry after ethanol exchange and drying in a vacuum oven. Then the MOF-808 concentration was adjusted into about 40 mg/mL, 80 mg/mL, and 160 mg/mL by removing or adding DI water after centrifugation.

To prepare the culture stock for MOF-808/BC hydrogel synthesis, the water above the slurry was exchanged three times with the same volume of bacterial suspension. After being vortexed for 10 min, the 40 mL of uniform slurry was carefully added into the Petri dish and cultured at 26° C. in static conditions for 5 days to get a non-floating hydrogel.

### Activation of the Hydrogel to Get MOF-808/BC Composite

The obtained hydrogel sample was washed in 500 mL DI water at 60° C. for 24 h three times to lyse the bacteria and remove the residual culture media from the hydrogel. To further remove the organics introduced into MOF pores during fermentation, the hydrogel was exchanged in 500 mL formic acid water solution (2 v/v %) at 60° C. overnight. The composite sample was exchanged in 100 mL ethanol (4 h×3 times) and then activated using supercritical CO₂ using a Tousimis Samdri-931 critical point dryer at a bleed rate of 1.0 mL/min. The MOF-808/BC composites, originated from the culture media with 40 mg/mL, 80 mg/mL, and 160 mg/mL MOF-808, were named MOF-808/BC-1, MOF-808/BC-2, and MOF-808/BC-3.

The UiO-66-NH₂/BC composite was synthesized using the same method and culture media with 80 mg/mL UiO-66-NH₂ used during fermentation. Pure BC aerogel was also prepared using the same method, while no MOF nanoparticles were added in the culture media during fermentation.

### Preparation of MOF-808 Coated PET as the Control

MOF-808 coated PET cloth, termed MOF-808/PET-1, was prepared as the control sample in catalysis and liquid absorption tests. To get MOF-808/PET-1, a piece of commercial PET cloth (5 cm×5 cm) was immersed in 10 mL MOF-808 slurry (200 mg/mL) in ethanol for 10 min and taken out with a weight pick up of 250%, then dried at room temperature in a hood overnight. The mass loading determined by ICP-OES was 50%.

### Liquid Phase Catalytic Hydrolysis of DMNP

Catalytic hydrolysis of DMNP experiments were carried out at room temperature and monitored by in situ ³¹P NMR. (Ma, K. et al., J. Am. Chem. Soc. 2019, 141, 15626-15633.) All activated composite catalysts were cut into tiny pieces (1 mm×1 mm×1 mm). For comparison of the catalytic efficiency of composites with different MOF loadings, all samples containing 1.5 μmol MOF-808 (2.2 mg) were added into a 3-dram vial. DMNP (4 μL, 25 μmol) was dissolved in 0.4 M N-ethylmorpholine solution (1 mL; 0.05 mL N-ethylmorpholine, 0.9 mL DI water/0.1 mL D₂O) and transferred into the vial. The sealed vial was vigorously vortexed for 10 s and magnetically stirred at 100 rpm. The simulant solution was filtered using a 200 nm syringe filter and transferred to an NMR tube for the 31p spectrum collection. The catalytic activity of pure BC without MOF and MOF-808/PET-1 was evaluated under identical conditions as a control.

### Liquid Uptake Testing

The MOF-808/BC composite samples' uptake of nerve agent simulant solution was evaluated by immersing the sample in 1 mL DMNP solution (4 μL dissolved in 0.4 M N-ethylmorpholine water solution) for 15 s. The composite sample was taken out and weight was measured to calculate the liquid pick up percentage. The liquid was extruded using a syringe with a filter within 2 min, and the conversion of the absorbed simulant was studied using the ³¹P NMR method. A MOF-808 coated PET, MOF-808/PET-1, was also tested as the control.

### Reusability and Storage Stability of MOF-808/BC Absorbent

To probe the reusability, the parallel MOF-808/BC-1 composite after the absorption test was washed with 10 mL water for 20 min, reactivated as described above, and reused in an absorption test. The absorption capacity and catalytic activity of the MOF-808/BC-1 composite after storage for 4 months in air also was tested to evaluate its durability.

### Preparation of MOF-808/BC-1/BPEIH Composite.

1 g of BPEI and 0.4 g of 1,3-butadiene diepoxide (50% in ethanol) were dissolved in 10 mL water. One piece of MOF-808/BC-1 (1 cm×1 cm) composite was immersed into the above solution for 10 min. (Ma, K. et al., 2021.) The composite was carefully taken out and incubated in an 8-dram glass vial at room temperature overnight to form crosslinked PEI hydrogel impregnated MOF-808/BC-1 (termed MOF-808/BC-1/BPEIH). The BC/BPEIH composite was also synthesized using the same method as a control.

Solid-State Hydrolysis of DMNP with MOF-808/BC-1/BPEIH Under Ambient Humidity

DMNP (4 μL, 25 μmol) was dropped onto the center of the MOF-808/BC-1/BPEIH composite (0.6 cm×0.6 cm×0.15 cm, containing 1.5 μmol MOF-808) in an uncapped vial, and then put into the humidifier oven at 50% relative humidity for a recorded time. (Ma, K. et al., 2021.) The samples were digested using 0.7 mL of D₂SO₄/DMSO-D₆ (15/100 V/V) and transferred to an NMR tube for a ³¹P NMR test.

### Stability Test on MOF-808/BC-1/BPEIH Composite

MOF-808/BC-1/BPEIH that had been in a sealed vial for 2 months was tested in solid state hydrolysis to evaluate the storage stability. To test the stability of the composite after exposure to high humidity, the composites were incubated in a humidifier oven at 100% relative humidity for 24 hours and solid-state hydrolysis was tested. To test the reusability of the composite catalyst, the parallel samples after one cycle of solid-state hydrolysis were washed with DI water for 30 min. After removing the excess water on the surface, the samples were tested again. To test the tolerance of the MOF-808/BC-1/BPEIH sample against sweat exposure, the composite was wetted with 200 μL of artificial sweat (pH=4.5) for 24 hours, and DMNP (4 μL) was added to the center of the above MOF-808/BC-1/BPEIH sample for a catalytic test. To test the catalytic performance of the sample after exposure to dense CO₂ and simulated gasoline atmosphere, 50 mg dry ice or 100 μL octane was added in a sealed container (1 L) loaded with MOF-808/BC-1/BPEIH samples. After standing for 24 h. the solid-state hydrolysis tests on the samples were conducted.

### GD Permeation Test

GD permeation testing was conducted in accordance with ASTM F739-12. A 1.5 in by 1.5 in swatch was sealed in a 1 in diameter glass permeation cell. A countercurrent air flow of 300 mL min⁻¹ at ˜2% RH was applied above and below the swatch. GD was fed to the swatch at a concentration of 300 mg m⁻³. The feed, retentate, and permeate concentrations were monitored using an Agilent 6890 gas chromatograph with a flame ionization detector. Tests were terminated when steady state was reached and the sum of the retentate and permeation concentrations equaled the feed concentration.

### Dose-Extraction Test

Dose-extraction tests were performed to probe each MOF-808/BC composite material's reactivity to GD. Samples were prepared by placing a measured mass of sorbent in a scintillation vial. The samples were then gently dried at 50° C. for 2 h. A humidification chamber with internal circulation was used to humidify the samples overnight at 50% relative humidity and 25° C. A dose of chemical agent was then delivered to the prepared vials at a ratio of 10 μL to 10 mg of MOF-808/BC composite. The vials were capped and allowed to stand for 24 h. Extraction was conducted using acetonitrile (1.5 mL) with the slurry mixture, which was then filtered and transferred to an autosampler vial. Analysis of the extract was performed using a GC with a mass spectrometer.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” can be only one or can mean “one or more.” Embodiments of the inventions consistent with either construction are covered.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method of making a bacterial cellulose-metal-organic framework composite, the method comprising:
preparing an aqueous fermentation medium comprising water, a cellulose-producing bacteria, a carbon source, and a nitrogen source;
adding metal-organic framework particles to the aqueous fermentation medium and fermenting the aqueous fermentation medium to form a hydrogel comprising the metal-organic framework particles embedded in a bacterial cellulose nanofiber network; and
converting the bacterial cellulose hydrogel having the metal-organic framework particles dispersed therein into an aerogel comprising the metal-organic framework particles embedded in a bacterial cellulose nanofiber network.

2. The method of claim 1, wherein the aqueous fermentation medium is free of organic solvent.

3. The method of claim 1, wherein fermenting the aqueous fermentation medium to form a hydrogel comprising the metal-organic framework particles embedded in a bacterial cellulose nanofiber network is carried out at a temperature of no greater than 40° C.

4. The method of claim 3, wherein the aqueous fermentation medium is free of organic solvent.

5. The method of claim 1, wherein the aerogel has a metal-organic framework particle loading of at least 70 wt. %, based on the combined weight of the bacterial cellulose nanofiber network and the metal-organic framework particles.

6. The method of claim 1, wherein the metal-organic framework particles comprise NU series metal-organic frameworks, UiO series metal-organic frameworks, or a combination thereof.

7. The method of claim 1, wherein the metal-organic framework particles are catalytic for the hydrolysis of an organophosphate compound.

8. The method of claim 1, further comprising applying a polymeric base to the aerogel.

9. The method of claim 8, wherein the polymeric base forms a crosslinked hydrogel that impregnates pores in the aerogel.

10. The method of claim 1, wherein converting the hydrogel into the aerogel comprises supercritical CO₂ drying of the hydrogel.

11. A bacterial cellulose-metal-organic framework composite comprising:
an aerogel comprising metal-organic framework particles embedded in a bacterial cellulose nanofiber network; and
a polymeric base on the aerogel, in the aerogel, or on and in the aerogel.

12. The composite of claim 11, wherein the polymeric base is a polyethyleneimine.

13. The composite of claim 11, wherein the polymeric base forms a crosslinked hydrogel that impregnates pores in the aerogel.

14. The composite of claim 11, wherein the metal-organic framework particles comprise NU series metal-organic frameworks, UiO series metal-organic frameworks, or a combination thereof.

15. The composite of claim 11, wherein the metal-organic framework particles are catalytic for the hydrolysis of an organophosphate compound.

16. A method of disabling a toxic agent comprising an organophosphate compound having a hydrolysable bond, the method comprising exposing the organophosphate compound to a composite in the presence of water, the composite comprising:
an aerogel comprising metal-organic framework particles that are catalytic for the hydrolysis of the organophosphate compound embedded in a bacterial cellulose nanofiber network; and
a polymeric base on the aerogel, in the aerogel, or on and in the aerogel,
wherein the metal-organic framework particles catalyze the hydrolysis of the hydrolysable bond.

17. The method of claim 16, wherein the polymeric base forms a crosslinked hydrogel that impregnates pores in the aerogel.
